(19) United States
(12) Patent Application Publication
(10) Pub. No.: US 2005/0197527 A1
(43) Pub. Date: Sep. 8, 2005

(54) Adjustable heart constraining apparatus and method therefore

(76) Inventor: Steven F. Bolling, Ann Arbor, MI (US)

Correspondence Address: HARNESS, DICKEY & PIERCE, P.L.C., P.O. BOX 828, BLOOMFIELD HILLS, MI, 48303, US

(21) Appl. No.: 10/793,469

(22) Filed: Mar. 4, 2004

(51) Int. Cl. A61F 2/00; A61F 13/00

(52) U.S. Cl. 600/37

(57) ABSTRACT

An apparatus and method for treating congestive heart disease and related cardiac complications, such as valvular disorders, including a constraining device placed on a target portion of the heart or placed over the pericardium. The constraining device may be an adjustable band system defining an area under which a dysfunctional valve is located. The band system is dimensioned and constructed to exert a desired local tension or compression to selectively constrain the target portion of the heart. The band system is adapted to be adjusted for each individual band on the heart to snugly conform to an extrapericardia geometry of the heart to constrain expansion of the underlying heart portion to a desired shape and configuration.

### FIELD OF THE INVENTION

The invention relates, in general, to heart constraining devices for effecting the cardiac output functions; and more particularly, to improvements in adjustable devices and methods of use to constrain the heart for treating congestive heart disease and related valvular dysfunction.

### BACKGROUND

The syndrome of heart failure is a common course for the progression of many forms of heart disease. Congestive heart disease is a progressive and debilitating illness. The disease is characterized by a progressive enlargement of the heart. Heart failure may be considered to be the condition in which an abnormality of cardiac function is responsible for the inability of the heart to pump blood at a rate commensurate with the requirements of the metabolizing tissues, or can do so only at an abnormally elevated filling pressure. There are many specific disease processes that can lead to heart failure with a resulting difference in pathophysiology of the failing heart, such as the dialatation of the left ventricular chamber. Etiologies that can lead to this form of failure include idiopathic cardiomyopathy, viral cardiomyopathy, and ischemic cardiomyopathy.

As the heart enlarges, it is forced to perform an increasing amount of work in order to pump blood with each heart beat. In time, the heart becomes so enlarged that the heart cannot adequately supply blood. An afflicted patient is fatigued, unable to perform even simple tasks, experiencing pain and discomfort. Further, as the heart enlarges, the internal heart valves cannot adequately close. This impairs the function of the valves and further reduces the heart's ability to supply blood. It may be desirable to enhance the valvular efficiency by using a constraining device targeting the dysfunctional heart valve. The constraining device may be adjustable for various reasons.

Causes of congestive heart disease are not fully understood. In some cases, the heart may enlarge to such an extent that the adverse consequences of heart enlargement continue after a viral infection has passed and the disease continues its progressively debilitating course.

Drug therapy treats the symptoms of the disease and may slow the progression of the disease. Presently, there may be no cure for congestive heart disease. Even with drug therapy, the disease may progress. Drugs are sometimes employed to assist in treating problems associated with cardiac dilation. For example, digoxin® increases the contractility of the cardiac muscle and thereby causes enhanced emptying of the dilated cardiac chambers. On the other hand, some drugs, for example, beta-blocking drugs, decrease the contractility of the heart and thus increase the likelihood of dilation. Other drugs including angiotension-converting enzyme inhibitors, such as enalopril®, help to reduce the tendency of the heart to dilate under the increased diastolic pressure experienced when the contractility of the heart muscle decreases. Many of these drugs, however, have side effects which make them undesirable for long-term use.

Presently, the only permanent treatment for congestive heart disease is heart transplant. To qualify, a patient must be in the later stage of the disease. Such patients are extremely sick individuals. Due to the absence of effective intermediate treatment between drug therapy and heart transplant, sick patients will have suffered terribly before qualifying for heart transplant. Further, after such suffering, the available treatment is unsatisfactory. Heart transplant procedures may be risky, invasive, and expensive.

Not surprising, substantial effort has been made to find alternative treatments for congestive heart disease. Recently, a new surgical procedure has been developed. Referred to as the Batista procedure, the surgical technique includes dissecting and removing portions of the heart in order to reduce heart volume. This is a radical new and experimental procedure subject to substantial controversy. Furthermore, the procedure is highly invasive, expensive, and commonly includes other expensive procedures (such as a concurrent heart valve replacement).

Cardiomyoplasty is a recently developed treatment for earlier stage congestive heart disease. In this procedure, the latissimus dorsi muscle (taken from the patient's shoulder) is wrapped around the heart and chronically paced synchronously with ventricular systole. Pacing of the muscles results in muscle contraction to assist the contraction of the heart during systole. While cardiomyoplasty has resulted in symptomatic improvement, the nature of the improvement is not understood.

Even though cardiomyoplasty has demonstrated symptomatic improvement, studies suggest the procedure only minimally improves cardiac performance. The procedure is highly invasive requiring harvesting a patient's muscle and an open chest approach (i.e., sternotomy) to access the heart. Furthermore, the procedure is expensive, especially those using a paced muscle. Such procedures require costly pacemakers. The cardiomyoplasty procedure is also complicated. For example, it is difficult to adequately wrap the muscle around the heart with a satisfactory fit. Also, if adequate blood flow is not maintained to the wrapped muscle, the muscle may necrose. The muscle may stretch after wrapping reducing its constraining benefits and is generally not susceptible to post-operative adjustment. Finally, the muscle may fibrose and adhere to the heart causing undesirable constraint on the contraction of the heart during systole.

In addition to cardiomyoplasty, mechanical assist devices have been developed as intermediate procedures for treating congestive heart disease. Such devices include left ventricular assist devices (LVAD) and total artificial hearts (TAH). A TAH includes a mechanical pump for urging blood flow from the left ventricle and into the aorta. An example of such is shown in U.S. Pat. No. 4,995,857 to Arnold. LVAD surgeries are still in U.S. clinical trials and not generally available. Such surgeries are expensive. The devices may be at risk of mechanical failure and may require external power supplies. TAH devices, such as the celebrated Jarvik heart, are used as temporary measures while a patient awaits a donor heart for transplant.

U.S. Pat. No. 5,702,343 to Alferness teaches a jacket to constrain cardiac expansion during diastole. The cardiac reinforcement device (CRD) provides for reinforcement of the walls of the heart by constraining cardiac expansion, beyond a predetermined limit, during diastolic expansion of the heart. The Alferness CRD, however, is not valve specific.

PCT International Publication No. WO 98/29041 discloses a cardiac constraint in a form of surfaces on opposite sides of the heart with the surfaces joined together by a cable through the heart or by an external constraint. U.S. Pat. No. 6,193,648 to Krueger teaches a heart constraining jacket having a flexible drawstring laced through the material with one end of the drawstring fixed in place and with the other end displaceable relative to the material. In response to a tension on the drawstring the material of the jacket may be bunched together, this is in response to relative movement between the ends of the drawstring. A spring-like releasable stay member is provided for holding the draw string in a fixed position relative to the jacket upon release of a pulling tension on the draw string. The stay member may then be releasable in response to a resumed pulling tension on the drawstring. The Krueger jacket, however, is not valve specific, and over the time, the heart constraining jacket may need to be adjusted. Thus, it is desirable to have an adjustable heart constraining device for treating congestive heart disease and related cardiac complications, such as valvular disorders by placing an adjustable constraining device on a target portion of the heart or placed over the pericardium.

### SUMMARY

According to various embodiments a heart constraining apparatus may selectively constrain a selected portion of a heart. The apparatus includes a member that may be positioned near the selected portion of the heart. A constraining mechanism is operable to engage the selected portion of the heart and is held near the selected portion of the heart with the member. An adjustment mechanism is operably connected to the constraining mechanism to adjust the constraining mechanism. The constraining mechanism selectively constrains the selected portion of the heart based at least upon the adjustment by the adjustment mechanism.

According to various embodiments a method for increasing the competency of a selected valve of a heart with a selectively constraining mechanism is disclosed. The method includes positioning the constraining mechanism relative to the selected portion of the heart. The selected portion of the heart is constrained to a first degree during positioning of the constraining mechanism. The selected portion of the heart may then be constrained to a second degree with the constraining mechanism. Constraining the selected portion of the heart to the second degree is substantially accomplished by selectively activating the constraining mechanism.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1A is a perspective view of a constraining device according to various embodiments;

FIG. 1B is a side elevational environmental view of a heart with the device of FIG. 1A in place;

FIG. 2A is a perspective view of a device according to various embodiments;

FIG. 2B is a side elevational environmental view of a heart with the device of FIG. 2A in place;

FIG. 3 is a perspective view of an implanted adjustable extrapericardium heart constraining element over the pericardium according to various embodiments;

FIG. 4 is a perspective view of an adjustable extrapericardium heart constraining band having a shape memory member;

FIG. 5 is a schematic representation of an adjustable member of the adjustable heart constraining band before and after an adjustment step; and

FIG. 6 is a perspective view of an adjustable extrapericardium heart constraining band having fluid expandable chambers.

### DETAILED DESCRIPTION OF VARIOUS EMBODIMENTS

The following description of various embodiments is merely exemplary in nature and is in no way intended to limit the invention, its application, or uses. A heart constraining assembly may be any appropriate member, such as a band, bands, nets, jackets, patches, or the like for constraining a specific portion or region of the heart from outside of the pericardium. Therefore, it will be understood that the following discussion is merely exemplary and not intended to be limiting.

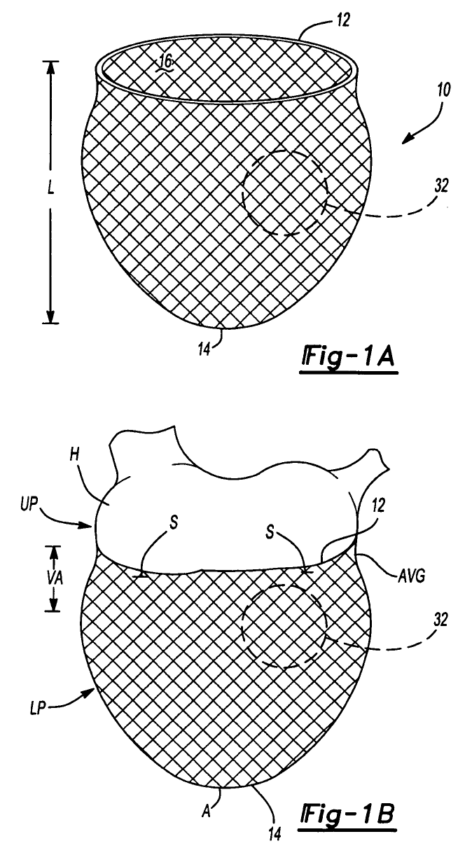

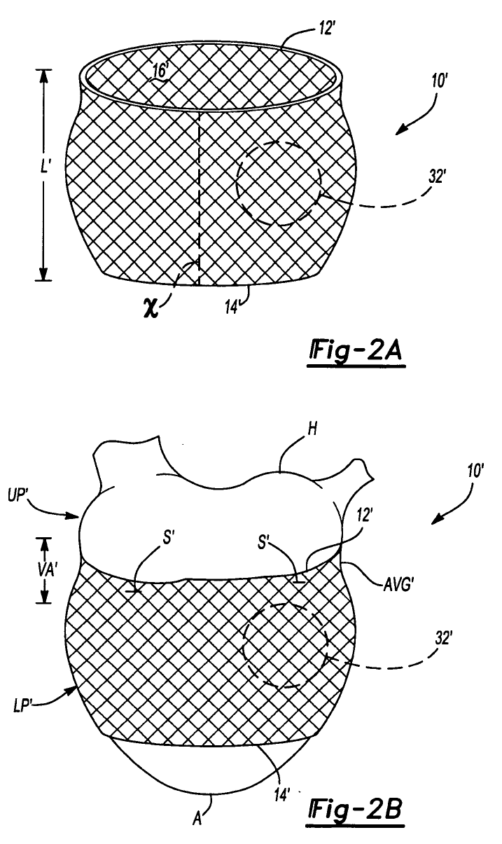

With reference to FIGS. 1A, 1B, 2A and 2B, a constraining assembly includes a jacket 10 of a flexible and biologically compatible material. The jacket 10 is an enclosed knit material having an upper end 12 and a lower end 14. The jacket 10 defines an internal volume 16 which is completely enclosed but for the upper end 12 being open. According to various embodiments, lower end 14 is closed. Various embodiments include an open lower end 14′ (FIGS. 2A and 2B) as well as an open upper end 12′. Upper ends 12, 12′ may be selected to be open for various reasons, such as ease of implantation and vein and/or vessel connections. Elements in common among various embodiments are numbered identically with the addition of an apostrophe to distinguish the FIGS. 2A and 2B from 1A and 1B.

The jacket 10 is dimensioned with respect to a heart H to be treated. Specifically, the jacket 10 is sized for the heart H to be constrained within the volume 16. The jacket 10 can be positioned around the heart H. The jacket 10 has a length L between the upper end 12 and the lower end 14, sufficient for the jacket 10 to constrain the lower portion LP of the heart H. The upper end 12 of the jacket 10 extends at least to the valvular annulus VA and further extends to the lower portion LP to constrain at least the lower ventricular extremities.

Because enlargement of the lower portion LP may be most troublesome, in various embodiments, the jacket 10 is sized so that the upper end 12 can reside in the A-V groove (AVG). Where it is desired to constrain enlargement of the upper portion UP of the heart H, the jacket 10 may be extended to cover the upper portion UP as well.

Various reasons exist for sizing the jacket 10 so that the upper end 12 terminates at the AVG. First, the AVG is a readily identifiable anatomical feature to assist a surgeon in placing the jacket 10. By placing the upper end 12 in the AVG, the jacket 10 may provide sufficient constraint at the valvular annulus VA. The AVG and the major vessels act as natural stops for placement of the jacket 10 while assuring coverage of the valvular annulus VA. Using such features as natural stops is particularly beneficial in minimally invasive surgeries where a surgeon's vision may be obscured or limited.

When the parietal pericardium is opened, the lower portion LP is free of obstructions for applying the jacket 10 over the apex A. If, however, the parietal pericardium is intact, the diaphragmatic attachment to the parietal pericardium inhibits application of the jacket over the apex A of the heart. In this situation, the jacket can be opened along a line X extending from the upper end 12′ to the lower end 14′ of the jacket 10′. The jacket 10′ can then be applied around the pericardial surface of the heart and the opposing edges of the opened line secured together after placement on the heart. Systems for securing opposing edges are disclosed in, for example, U.S. Pat. No. 5,702,343 entitled “Cardiac Reinforcement Valve”, the entire disclosure of which is incorporated herein by reference. The lower end 14′ can then be appropriately secured to the diaphragm or associated tissues using, for example, sutures, staples, etc.

In various embodiments, the lower end 14 is closed and the length L is sized for the apex A of the heart H to be received within the lower end 14 when the upper end 12 is placed at the AVG. In various embodiments, the lower end 14′ is open and the length L′ is sized for the apex A of the heart H to protrude beyond the lower end 14′ when the upper end 12′ is placed at the AVG. The length L′ is sized so that the lower end 14′ extends beyond the lower ventricular extremities such that in both of the jackets 10, 10′ the myocardium surrounding the ventricles is in direct opposition to the material of the jacket 10, 10′. Such placement is desirable for the jacket 10, 10′ to present a constraint against enlargement of the ventricular walls of the heart H.

After the jacket 10 is positioned on the heart H, as described above, the jacket 10 may be secured to the heart. As discussed, the jacket 10, 10′ may be implanted through a substantially open procedure or a generally minimally invasive procedure. Regardless, the jacket 10, 10′ may be placed near the heart H for various reasons. The jacket 10 may be secured to the heart H using any appropriate means, such as through sutures. The jacket 10 may be sutured to the heart H at selected locations, such as suture locations S circumferentially spaced along the upper end 12. While a surgeon may elect to add additional suture locations to prevent shifting of the jacket 10, after placement, the number of suture locations S may be limited so that the jacket 10 does not restrict contraction of the heart H during systole.

With reference to FIGS. 1A, 1B and 4, a shape memory member 38 may be used with the jacket 10, comprise the jacket 10, or be used with other members. The shape memory portion may refer to any portion of the construction material for the adjustable extrapericardium heart constraining element, wherein the shape memory member can be pre-shaped at a non-deployment state and regain its shape at a deployed state or vice versa. An exemplary shape memory metal is Nitinol®, as shown in U.S. Pat. No. 6,077,293 to Tu, et al., incorporated herein by reference. In principle, the shape memory materials, such as Nitinol® have a preshape and a shape transition temperature. The shape memory material retracts to its preshape and causes an expandable strand to collapse when the shape memory Nitinol® member is heated to above the shape transition temperature at a retracted state. The preferred shape transition temperature is about a few degrees above the body temperature; it may range from 38° C. to 45° C. or higher. Other temperature ranges, however, such as 37° C. or less (hypothermal) and 38° C. or above (hyperthermal) may equally be applicable. The shape memory material may be constructed in a wire, coil, or other appropriate form as part of the adjustable extrapericardium heart constraining device assembly, such as the jacket 10, a band, bands or the like, as discussed herein.

The material for the shape memory member 38 may also be any appropriate material, such as a plastic reported by White and Ward (pp. 635-640, Materials Research Society Symposium Proceedings vol. 110), the entire contents of which are incorporated herein by reference. The softenability and the shape memory properties of Calomer™ thermoplastics depend upon the glass transition temperature (Tg) of the polymer. At temperatures below the Tg, molecular movement is restricted and the material is rigid and stiff. At temperatures at or above the Tg, the free volume for thermal motion of the molecular backbone increases and the material behaves as a rubbery or high viscosity liquid. For biomedical devices the Tg of interest may be about room temperature or above. Other ranges may be similar to that of Nitinol®.

If softenable shape memory polymers and particulate fillers are combined on a twin-screw extruder, palletized thermoplastic compounds can be produced that are suitable for a variety of biomedical applications (for example, catheters, wound closures, endotracheal tubes, threads, bands, etc). The extent of modulus change exhibited by a particular compound can be varied through the hard segment/soft segment range in the polymer. A permanent shape can be set in the original conversion process or by post forming in an oven. Temporary shapes can be set by cord forming or by heating above the lower Tg and then quenching in the desired shape. Softenability without shape recovery is achieved by omitting the temporary shape forming step and making the temporary and permanent shapes identical. The selection of Tg may be a temperature slightly above or below the about body temperature, such as 1° C. or more from the reference body temperature.

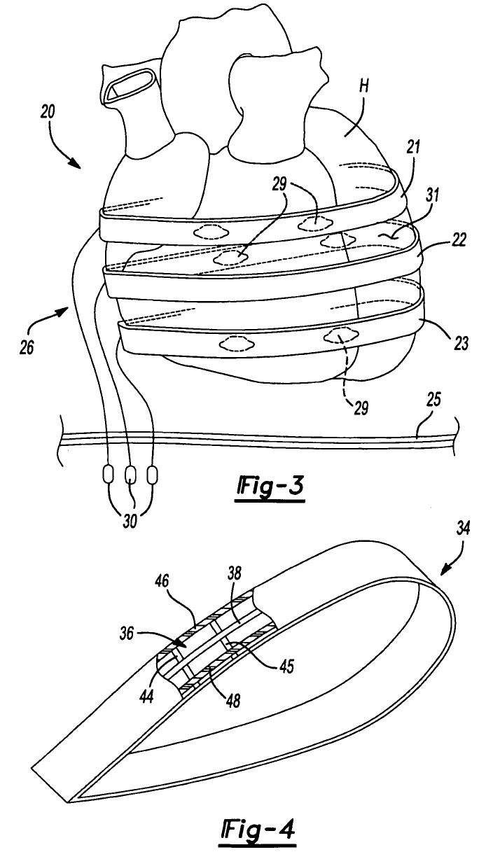

With reference to FIG. 3, an adjustable heart constraining assembly 20 is illustrated. According to various embodiments, the adjustable heart constraining assembly includes an extrapericardium band 21 or may include a plurality of bands 21, 22, 23 that may be wrapped over the outside of the pericardium of the heart H. The adjustable bands 21, 22, or 23 may be placed at about the level of a diaphragm 25, or other appropriate level. The bands 21, 22, 23 have adequate length effective for wrapping around the pericardium and have an appropriate width, but may be about 1 cm to about 60 cm wide. The bands 21, 22, 23 may be placed by an open chest operation or a minimally invasive manner through openings in the diaphragm 25.

The extrapericardium bands 21, 22, 23 are adjustable by a constraining mechanism or device 26. The constraining mechanism 26 may shorten the inner circumference of the adjustable bands 21, 22, 23 so that the pericardium is constrained by the adjustable bands 21, 22, 23. The constraining mechanism 26 may include the shape memory material retraction, expandable chambers, a drawstring, or the like to shorten the inner circumference of the adjustable band. The constraining mechanism 26 may apply to the adjustable bands 21, 22, 23, evenly along the band length/width, or selectively at a portion of the band length or width.

The constraining mechanism 26 may include an expandable chamber 29, or may include a plurality of the expandable chambers 29 that may be positioned on any one or all of the bands 21, 22, 23. The expandable chambers 29 may be expanded in any appropriate manner such as inflation with a selected fluid, such as a sterile air, saline, and other appropriate fluids. The fluids may be provided to the expandable chambers 29 from a reservoir 30, or a plurality of reservoirs 30 may be provided. The reservoirs 30 may be implanted when the bands 21, 22, 23 are implanted or implanted during a subsequent procedure. The reservoirs 30 may be provided subcutaneously for ease of supplying a selected amount of fluid to the expandable chambers 29 or may be provided externally as selected. Regardless, at a selected time, the expandable chambers 29 may be expanded to decrease an internal circumference or diameter of one or a plurality of the bands 21, 22, 23 for various reasons.

For example, the congestive heart disease may proceed or progress to an amount that requires a greater amount of constriction on the heart H. Therefore, the internal diameter or circumference of the bands 21, 22, 23 may be decreased by inflating the expandable chambers 29. It will be understood that the expandable chambers 29 may be provided on all or a selected number of the plurality of bands 21, 22, 23. They may also be provided on any appropriate member that may be positioned near the heart H. For example, the expandable chambers 29 may be provided at selected positions within the jacket 10, 10′ illustrated in FIGS. 1A-2B. Therefore, providing or locating the expandable chambers 29 on the bands 21, 22, 23 is merely exemplary.

In addition, the reservoirs 30 may be provided in any appropriate location. For example, ports may be provided through the dermis and the reservoirs 30 provided during a subsequent procedure to fill the expandable chambers 29. Therefore, the reservoirs 30 need not be provided subdermally within a patient. Nevertheless, if the reservoir chambers 30 are provided in the patient, they may be activated in any appropriate manner. For example, a wireless command may be used to activate the reservoirs 30 to fill the expandable chambers 29 a selected amount. Alternatively, the reservoirs 30 may be manually or mechanically activated to expand the expandable chambers 29. Nevertheless, it will be understood that the reservoirs 30 may be refilled from an external location to allow for filling the expandable chambers 29 to any appropriate amount. In addition, the expandable chambers 29 may be filled any selected amount to provide a selected degree of constriction on the heart H. Moreover, as discussed herein, each of the expandable chambers 29 may be individually expanded for maximum selectivity. Alternatively, all may be filled concurrently.

FIG. 4 shows an adjustable band 34 comprising constraining mechanism 36 that includes the shape memory member 38. For example, the constraining mechanism 36 may be associated with the shape memory member 38 and be bounded by members 44 and 45. The mechanism 36 may also contain some inactive supporting fibers 46, 48 or other threads which are flexible but noncompliant. For illustrative purposes, the constraining mechanism 36 may include an appropriate shape memory member 38, made of a selected material such as a shape memory Nitinol®, a shape memory plastic, or combinations thereof. According to the shape memory principles, after the temperature of a shape memory passes its own thermal transition temperature, a dimension, such as length of the constraining mechanism 36 changes.

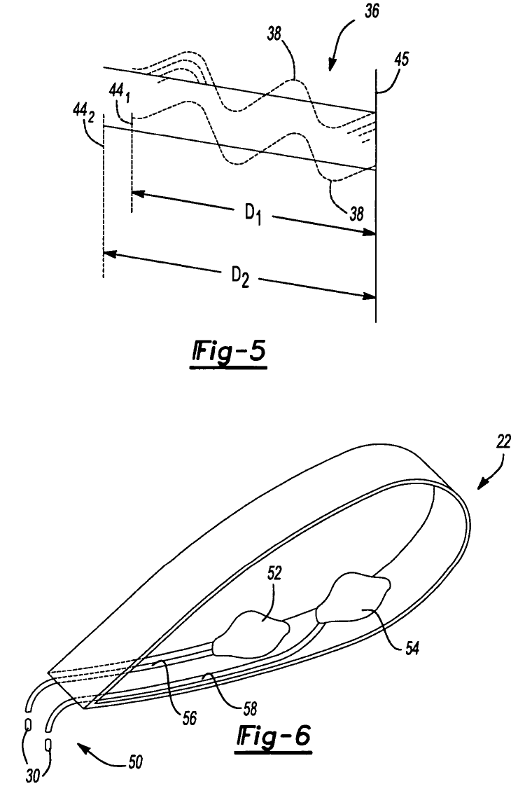

With reference to FIG. 5, a schematic view of the constraining mechanism 36 is illustrated. The constraining mechanism 36, including the shape memory material may change its shape depending upon a temperature of the material. The adjustment shape memory member 38 of the constraining mechanism 36 includes a first length or distance D₁ at a first temperature. The adjustment shape memory member 38 may then change to a second length or distance D₂ when its temperature changes. Specifically, members 44, 45 may be separated by the distance D₁ such as the distance between members 44₁ and 45. Then when the adjustment shape memory member 38 changes, such as due to a temperature change, the distance D₂, the distance between members 44₂ and 45, may be achieved. This may, among other things, effectively decrease the internal diameter or circumference of the band 34 for various reasons.

The adjustable constraining mechanism 36 may be triggered or activated by a selected force, such as a heat source or cryogenic source, such as instrument made of Peltier effects at any time period post-placement. Also, the constraining mechanism 36 may be irradiated with a particular energy to allow it to expand or contract at a selected rate or amount. The thermal source for activating the shape memory shift for the shape memory member 38 may be conductive means such as radio frequency monopolar heat generation, such as that disclosed in U.S. Pat. No. 6,077,298, a wire having Peltier effects, or non-invasive radiation means such as ultrasound, electromagnetic or paramagnetic. Providing a heat or thermal change to cause the expansion or contraction of the constraining mechanism 36 is merely exemplary.

The adjustment, whether through inflation or the shape memory member 38, may accommodate the heart shrinkage, heart aging, or heart enlargement over time. Therefore, the above-described adjustable step or mechanism may include either tightening or loosening the constraining mechanism 36.

To permit the band 34 to be easily placed on the pericardium, the length and shape of the band 34 are larger than the target portion during diastole. (Although band 34 is discussed here, it will be understood any band may be so used.) So sized, the band 34 may be easily slipped around and/or over the pericardium. Once placed, the length and shape of the band 34 may be adjusted for the band 34 to snugly conform to the external geometry of the pericardium during diastole. Such sizing is easily accomplished in a fiber or other material that is used for construction of the band 34. For example, the fibers 46, 48 may be about 70 Denier polyester. It will be understood that any appropriate material may be used including polytetrafluoroethylene (PTFE), expanded PTFE (ePTFE), polypropylene, nature fibers, arid stainless steel or other metal. The bands 21, 22, 23 may also include such fibers to assist in proper placement and sizing.

Returning reference to FIG. 3, an open area 31 between the extrapericardium bands allows for electrical connection between the heart H and surrounding tissue for passage of electrical current to and from the heart H. For example, although the band 21 may be an electrical insulator, the open area 31 is sufficiently electrically permeable to permit the use of transchest defibrillation of the heart H. Also, the open, flexible construction permits passage of electrical elements (e.g., pacer leads) through the open area 31 between the bands 21, 22, 23. Additionally, the open band construction permits other procedures, e.g., coronary bypass, to be performed without removal of the bands 21, 22, 23.

The bands 21, 22, 23 may also be connected to each other and form a net-like structure. Therefore, a webbing or other material may interconnect a plurality of bands, such as between the bands 21 and 22 and between the bands 22 and 23. In addition, the bands 21, 22, 23, or any appropriate number, either more or less than three bands, may be used with the constraining mechanisms illustrated in FIGS. 1A-2B. Therefore, the bands 21, 22, 23 may be used alone or in conjunction with another constraining apparatus.

Continuing reference to FIG. 3 and with additional reference to FIG. 6, at least one of the adjustable extrapericardium heart constraining bands 22 may include a constraining mechanism 50. The constraining mechanism 50 may be provided in any of the bands 21, 22, 23 and may be provided in addition to the constraining mechanism 36, including the shape memory member 38, or in place of the constraining mechanism 36. Therefore, it will be understood that the constraining mechanism 50 may be used in addition to or in place of the constraining mechanism 36 for various reasons.

The constraining mechanism 50 may include a first fluid expandable chamber 52, that may be inflated with a fluid, and/or may include a second fluid expandable chamber 54, that may also be inflated with a fluid, or any appropriate number of fluid expandable chambers. Nevertheless, the constraining mechanism 50 may include any appropriate number of the fluid expandable chambers 52, 54 at a selected position along the band 22. For example, the fluid expandable chambers 52, 54 may be placed on the inside of the band 22 or on an exterior surface of the band 22. Although the exact location of the fluid expandable chambers 52, 54 on the adjustable band 22 may be determined using any appropriate reason or process, it may be selected for various anatomical and medical reasons. For example, the location of the fluid expandable chambers 52, 54 may be determined or positioned due to a need to effectively constrain the underlying heart valve.

The first fluid expandable chamber 52, and the second fluid expandable chamber 54, if provided, may be connected to the fluid reservoir 30 (shown in FIGS. 3 and 6) through an appropriate supply line 56. The single supply line 56 may be provided to each of the fluid expandable chambers 52, 54 or a plurality of supply lines, such as a second supply line 58, may be provided to any appropriate number of the fluid expandable chambers 52, 54. Therefore, each of the fluid expandable chambers 52, 54 may be operated substantially independently or as a group.

As discussed above, the fluid reservoir 30 may be provided substantially subcutaneously or externally to the body. Therefore, the fluid reservoir 30 may be implanted with the band 22 or may be provided during a period of selectively adjusting the adjustable band 22. Regardless, the line or lines 56, 58 may allow for either independent or mutual filling of the selected fluid expandable chambers 52, 54 at a selected time. The fluid used for inflating the fluid expandable chambers 52, 54 may be any appropriate fluid. For example, the fluid may include saline, heparinized saline, therapeutic fluid, anti-inflammatory fluid, angiogenic fluid, anti-angiogenic fluid, anti-proliferative fluid, gases, and growth factor solution.

The constraining mechanism 36, according to various embodiments, may selectively improve a selected heart valve competency. It may selectively constrain pericardium surrounding a selected heart valve. The adjustable heart constraining element may be an adjustable band or bands placed over the pericardium of the heart H or a patch mounted around the epicardial wall over the selected heart valve. Therefore, the bands 21, 22, 23 or any appropriate number of bands may be fixed relative to the heart H for selectively constraining a selected portion of the heart H, such as a selected valve. Therefore, a selected portion or valve of the heart H may be constrained substantially individually to increase competency of that portion without constraining the entire heart H.

Alternatively, or in addition to the bands 21, 22, 23, a partial band or patch member 32, 32′ may be fixed relative to a selected portion of the heart H. The patch member 32, 32′ may include the constraining mechanism 36 to constrain a selected area of the heart H. Therefore, rather than providing a band to encircle a selected portion of the heart H, the patch member 32, 32′ simply provides a selected area that is locally constrained.

In addition, the constraining mechanisms 36, 50 may be activated at a selected time. For example, the shape memory member 38 may be activated, thermally or through an irradiation, to expand or contract to select a particular tension or constraining amount to be applied to the selected area. Likewise, the fluid expandable chambers 52, 54 may be inflated at the selected time to provide a selected constraint in a selected area. Therefore, the constraining mechanisms 36, 50 need not be activated maximally at a single time and may be altered over a selected period of time.

In addition, the adjustable bands 21, 22, 23 may be implanted or positioned relative to the heart H and later adjusted using the adjusted constraining mechanisms 36, 50. In addition, the adjustable bands may be implanted in addition to or with the jacket 10, 10′ as described above. For example, the expandable chambers 52, 54 may be incorporated in the jacket 10, 10′. Alternatively, the jacket 10, 10′ may be implanted in addition to at least one of the bands 21, 22, 23. The constraining mechanisms 36, 50 may be provided with any appropriate portion to be held in place relative to the heart H.

As briefly discussed above, the bands 21, 22, 23 may be provided in a substantially specific location, such that they may constrain a selected valve, rather than the entire heart H. In addition, the bands 21, 22, 23 or a selected portion thereof may be formed of a substantially electrically conductive material. If the bands 21, 22, 23 or a portion thereof are formed of a substantially electrically conductive material, the bands 21, 22, 23 may be used as a stimulating mechanism. Therefore, the bands 21, 22, 23 or a selected portion thereof may be provided at a specific location to stimulate a selected portion of the heart H at a selected time. A defibrillator, pace maker or similar device may be implanted with the bands 21, 22, 23 or leads may be provided relative to the bands 21, 22, 23 for external stimulation. Also, the stimulation may provide an effective constraining at the heart H rather than a mechanical constraining.

In addition, the constraining mechanisms 36, 50 may be provided to any appropriate portion of the heart H. For example, the constraining mechanisms 36, 50 may be provided relative to the venous valve to selectively constrain the venous valve. Therefore, any of the appropriate valves in the heart H may be constrained by positioning the constraining mechanisms 36, 50 or one or more of the bands 21, 22, 23 relative to a selected portion of the heart H.

In addition, although the constraining mechanisms 36, 50 are illustrated and discussed having a shape memory member 38 or an expandable chamber 29, it will be understood that any appropriate mechanism may be provided for the constraining mechanism. Providing a shape memory member 38 or/with an expandable chamber 29 is merely exemplary and not intended to limit the disclosure. Furthermore, the constraining mechanisms 36, 50 may be provided on one, two, or any number of bands and is not limited to three, more than three, or less than three. Therefore, the constraining mechanisms 36, 50 may be provided in any appropriate numbers or bands or patches and positioned relative to the heart H for selected reasons. Only providing the bands 21, 22, 23 is merely exemplary and not intended to limit the disclosure in anyway.

The description of the invention is merely exemplary in nature, and thus, variations that do not depart from the gist of the invention are intended to be within the scope of the invention. Such variations are not to be regarded as a departure from the spirit and scope of the invention.

1. A heart constraining apparatus to selectively constrain a selected portion of a heart, the apparatus comprising:
a member positionable near the selected portion of the heart;
a constraining mechanism operable to engage the selected portion of the heart and substantially held near the selected portion of the heart with said member; and
an adjustment mechanism operably connected to said constraining mechanism to adjust said constraining mechanism;
wherein said constraining mechanism selectively constrains the selected portion of the heart based at least upon the adjustment by said adjustment mechanism.

2. The apparatus of claim 1, wherein said constraining mechanism includes at least one of a fluid expandable portion, a shape memory portion, an electrical stimulation portion, and combinations thereof.

3. The apparatus of claim 2, wherein said fluid expandable portion may expand from a first size to a second size;
wherein said second size operably decreases a selected dimension of said member.

4. The apparatus of claim 3, wherein said selected fluid includes at least one of a saline, a sterile saline, a growth factor, an anti-inflammatory fluid, an anti-angiogenic fluid, an angiogenic fluid, an anti-proliferative fluid, and combinations thereof.

5. The apparatus of claim 2, wherein said shape memory portion includes at least one of a shape memory metal, shape memory plastic, and combinations thereof.

6. The apparatus of claim 5, wherein said shape memory portion is selectable between a first size and a second size;
wherein said second size operably decreases a selected dimension of said member.

7. The apparatus of claim 1, wherein said constraining mechanism is adjustable by said adjustment mechanism at a time after positioning said member relative to the selected portion of the heart;
wherein said adjustment mechanism may operate subdermally or transdermally to adjust said constraining mechanism.

8. The apparatus of claim 1, wherein said adjustment mechanism includes a fluid reservoir positioned substantially subdermally;
wherein a fluid may be moved from said fluid reservoir to said constraining mechanism to change a selected dimension of said member.

9. The apparatus of claim 1, wherein said member and said constraining mechanism are positioned on a selected portion of the heart to operably constrain substantially only the selected portion of the heart.

10. The apparatus of claim 9, wherein the selected portion of the heart to be constrained includes at least one of a mitral valve, an aortic valve, a tricuspid valve, and combinations thereof.

11. The apparatus of claim 1, further comprising an electrical probe;
wherein said electrical probe is operably associated with said member to provide a selected electrical stimulation to the selected portion of the heart.

12. The apparatus of claim 1, wherein said member includes at least one of an extendable band, a jacket, a substantially non-extendable band, and combinations thereof.

13. A method for increasing the competency of a selected valve of a heart with a constraining mechanism, the method comprising:
positioning the constraining mechanism relative to the selected portion of the heart;
constraining the selected portion of the heart during positioning of the constraining mechanism to a first degree; and
constraining the selected portion of the heart after positioning the constraining mechanism to a second degree;
wherein constraining to said second degree is substantially accomplished by selectively activating the constraining mechanism.

14. The method of claim 13, wherein constraining the selected portion of the heart includes at least one of elastically constraining, inflating a selected bladder, expanding a shape memory material, electrically stimulating the selected portion of the heart, and combinations thereof.

15. The method of claim 14, wherein inflating a selected bladder includes providing a selected fluid to the bladder to expand the bladder from a first size to a second size.

16. The method of claim 15, further comprising:
implanting a fluid reservoir operably connected to said constraining mechanism substantially when positioning the constraining mechanism; and
moving a selected volume of said fluid from said fluid reservoir to the selected bladder.

17. The method of claim 14, wherein expanding a shape memory material includes at least one of heating, irradiating, and combinations thereof;
wherein expanding the shape memory material selectively constrains the selected portion of the heart.

18. The method of claim 13, wherein positioning the constraining mechanism includes positioning the constraining mechanism relative to a selected portion of the heart to selectively constrain substantially only the selected portion of the heart.

19. The method of claim 18, wherein the selected portion of the heart includes at least one of an aortic valve, a mitral valve, a tricuspid valve, and combinations thereof.

20. The method of claim 13, wherein positioning the constraining mechanism relative to a selected portion of the heart includes:
providing a band relative to the selected portion of the heart;
sizing said band to said first degree to constrain said selected portion of the heart to said first degree; and
fixing said band relative to the selected portion of the heart.

21. The method of claim 20, wherein constraining the selected portion of the heart to said second degree includes, after positioning the band and constraining the heart to said first degree, constraining the heart to said second degree by inflating a selected bladder, expanding a shape memory material, electrically stimulating, and combinations thereof.

22. The method of claim 13, further comprising:
stimulating the selected portion of the heart;
wherein stimulating the selected portion of the heart at least partially constrains the selected portion of the heart to said second degree.

23. The method of claim 13, wherein positioning the constraining mechanism includes positioning a member over a pericardium of the heart.

24. The method of claim 13, wherein constraining the selected portion of the heart to said second degree includes reducing a selected dimension of the constraining mechanism to apply a selected force to the selected portion of the heart to increase the selected valve competency.

25. The method of claim 13, wherein positioning the constraining mechanism relative to a selected portion of the heart includes:
providing a jacket relative to the selected portion of the heart;
sizing said jacket to said first degree to constrain said selected portion of the heart to said first degree; and
fixing said jacket relative to the selected portion of the heart.

26. The method of claim 25, wherein constraining the selected portion of the heart to said second degree includes, after positioning the jacket and constraining the heart to said first degree, constraining the heart to said second degree by inflating a selected bladder, expanding a shape memory material, electrically stimulating the selected portion of the heart, and combinations thereof.

27. A constraining apparatus to selectively and variably constrain the heart, the constraining apparatus comprising:
a sizable band to surround a selected portion of the heart; and
a constraining mechanism operable to interact with said sizable band;
wherein said sizable band is positioned to selectively surround a selected portion of the heart at a first time to apply a first constraining force to the heart;
wherein said constraining mechanism is activated to interact with said sizable band at a second time to apply a second constraining force to the heart.

28. The constraining apparatus of claim 27, wherein said sizable band is an extrapericardium sizable band substantially positionable on an exterior portion of the heart to apply said first constraining force to the heart.

29. The constraining apparatus of claim 27, wherein said constraining mechanism includes at least one of an expandable chamber, a shape memory material, an electrically conductive member, and combinations thereof.

30. The constraining apparatus of claim 29, further comprising a fluid reservoir;
wherein a fluid volume from said fluid reservoir is provided to said constraining mechanism to alter a selected dimension of said sizable band to apply said second constraining force to the heart.

31. The constraining apparatus of claim 29, further comprising an extradermal thermal source to activate said shape memory material to alter a dimension of said sizable band to provide said second constraining force to the heart.

32. The constraining apparatus of claim 27, wherein said sizable band selectively surrounds a selected portion of the heart including at least one of an aortic valve, a tricuspid valve, a mitral valve, and combinations thereof.

33. The constraining apparatus of claim 32, wherein said second constraining force substantially improves a competency of the at least one of said mitral valve, said aortic valve, said tricuspid valve, and combinations thereof.

34. The constraining apparatus of claim 27, further comprising an electrical stimulation lead;
wherein said electrical stimulation lead is operable to at least one of selectively constrain the heart, selectively defibrillate the heart and selectively pace the heart.

35. The constraining apparatus of claim 27, wherein said sizable band and said constraining mechanism are operable to allow an electrical stimulation to reach the heart after positioning of said sizable band and said constraining mechanism.

36. A constraining apparatus to selectively and variably constrain the heart, the constraining apparatus comprising:
a sizable jacket to surround a selected portion of the heart; and
a constraining mechanism operable to interact with said sizable jacket;
wherein said sizable jacket is positioned to selectively surround a selected portion of the heart at a first time to apply a first constraining force to the heart; and
wherein said constraining mechanism is activated to interact with said sizable jacket at a second time to apply a second constraining force to the heart.

37. The constraining apparatus of claim 36, wherein said constraining mechanism includes at least one of an expandable bladder, an electrical stimulate-able member, a shape memory member, and combinations thereof;
wherein said constraining mechanism is operable to change a pressure applied to a selected portion of the heart to variably constrain the heart.

38. The constraining apparatus of claim 37, wherein said constraining mechanism is activated at a time after said constraining mechanism is positioned relative to the heart.

39. The constraining apparatus of claim 38, further comprising:
a fluid reservoir operably connected to said expandable bladder;
wherein a fluid is moveable from said fluid reservoir to said expandable bladder to variably constrain the heart.
